(19) United States
(12) Patent Application Publication
(10) Pub. No.: US 2022/0133550 A1
(43) Pub. Date: May 5, 2022

(54) Diaper adapted to mount a wetness notifying device

(71) Applicant: I-Ding Medical Equipment Co., Ltd., Kaohsiung (TW)

(72) Inventor: Hung Chi Chen, Kaohsiung (TW)

(21) Appl. No.: 17/483,457

(22) Filed: Sep. 23, 2021

(30) Foreign Application Priority Data
Oct. 29, 2020 (TW) 109214261

(51) Int. Cl. A61F 13/42; A61F 13/496

(52) U.S. Cl. CPC A61F 13/42; A61F 2013/424; A61F 13/496

(57) ABSTRACT

A diaper (7) adapted to mount a wetness notifying device (10) includes a main body (70). The main body (70) includes an inner surface (71) and an outer surface (72), and is formed with a through hole (73) extending from the inner surface (71) to the outer surface (72). The wetness notifying device (10) includes a moisture detector (22) disposed on a side of the inner surface (71), and a transmitting module (23) disposed on a side of the outer surface (72). The wetness notifying device (10) is mounted to the diaper (7) in a manner that a transmission cable (28) is threaded through the through hole (73) and electrically connects the moisture detector (22) to the transmitting module (23), and that the moisture detector (22) is disposed at a position near an external urethral orifice of a user (8) wearing the diaper (7).

### FIELD OF THE INVENTION

The disclosure relates to a diaper, and more particularly to a diaper adapted to mount a wetness notifying device.

### BACKGROUND OF THE INVENTION

Referring to FIG. 1, Indian Patent Application No. 201614010143 discloses a wetness notification system including a wetness notifying device 1 and a receiving unit 16. The wetness notifying device 1 is reusable, and is configured to clip onto a conventional diaper 17 used to absorb urine. The wetness notifying device 1 sends a notification signal to the receiving unit 16 when it detects that the conventional diaper 17 becomes wet due to urination.

The wetness notifying device 1 includes a moisture detector (not shown), a transmitting module 11 (containing a processor, a wireless transmitter, a battery, etc., all not shown) capable of sending a wireless signal to the receiving unit 16, a transmission cable 14, a clip 13, a mounting seat 12 and a positioning member 15. The receiving unit 16 includes a light signal 161 and a speaker 162.

The clip 13 is inserted into the mounting seat 12, and the moisture detector is mounted to the mounting seat 12. The transmission module 11 is detachably secured on the clip 13, and the transmission cable 14 extends above and over the positioning member 15 while connecting the transmission module 11 to the moisture detector (not shown). The moisture detector is disposed on an inner side of the conventional diaper 17 via the clip 13 clipping to an edge of the conventional diaper 17.

When a user wears the conventional diaper 17 and urinates, which wets the conventional diaper 17, the moisture detector will detect wetness and send a notification signal via the transmission cable 14 to the transmission module 11. The transmission module 11 will wirelessly transmit the notification signal to the receiving unit 16. Upon receiving the notification signal, the receiving unit 16 outputs a notification (e.g., light outputted by the light signal 161 or voice outputted by the speaker 162) to notify a caregiver, so that the caregiver may change the conventional diaper 17. The receiving unit 16 may be carried by the caregiver or placed in a particular place such as a medical station, where the caregiver may be alerted by the notification from the receiving unit 16.

Since the wetness notifying device 1 is attached to the conventional diaper 17 via the clip 13 clipping to an edge of the conventional diaper 17 near the groin of the user, the clip 13 and the positioning member 15 will be in direct contact with the user's skin, which may cause discomfort to the user. In addition, the wetness notification system is limited by the wetness notifying device 1 only being able to be attached to the edge of the conventional diaper 17 near the groin of the user. Therefore, the wetness notification device 1 is positioned at least some distance away from an external urethral orifice of the user, consequently resulting in slower and less accurate detection by the moisture detector. These limitations cause the wetness notification system to fall short of its intended purpose of providing swift and accurate notification.

### BRIEF SUMMARY OF THE INVENTION

Therefore, an object of the disclosure is to provide a diaper and a diaper assembly that can alleviate at least one of the drawbacks of the prior art.

According to one aspect of the disclosure, a diaper is adapted to mount a wetness notifying device. The wetness notifying device includes a moisture detector configured to detect surrounding moisture and to output a notification signal when the surrounding moisture detected thereby exceeds a threshold moisture level, a transmission cable, and a transmitting module that is electrically connected to the moisture detector through the transmission cable for receiving the notification signal outputted by the moisture detector and that is configured to wirelessly transmit the notification signal. The transmission cable has one end fixedly connected to the moisture detector, and the other end removably connected to the transmitting module.

The diaper includes a main body that is adapted to be worn as underpants by a user, that includes an inner surface and an outer surface, and that is formed with a through hole extending from the inner surface to the outer surface. The through hole is configured to allow the wetness notifying device to be mounted to the diaper in a manner that the moisture detector is disposed on a side of the inner surface and the transmitting module is disposed on a side of the outer surface, that the other end of the transmission cable is threaded from the inner surface through the through hole and emerges out on the outer surface and is connected to the transmitting module, and that the moisture detector is at a position near an external urethral orifice of the user wearing the diaper.

According to another aspect of the disclosure, a diaper assembly includes a diaper and a wetness notifying device.

The diaper includes a main body that is adapted to be worn as underpants by a user. The main body includes an inner surface and an outer surface, and is formed with a through hole extending from the inner surface to the outer surface.

The wetness notifying device includes a moisture detector, a transmission cable and a transmitting module. The moisture detector is disposed on a side of the inner surface at a position near an external urethral orifice of the user wearing the diaper. The moisture detector is configured to detect surrounding moisture and to output a notification signal when the surrounding moisture detected thereby exceeds a threshold moisture level. The transmission cable is threaded through the through hole. The transmitting module is disposed on a side of the outer surface, is electrically connected to the moisture detector through the transmission cable for receiving the notification signal outputted by the moisture detector, and is configured to wirelessly transmit the notification signal. The transmission cable has one end fixedly connected to the moisture detector and the other end that is removably connected to the transmitting module, and that is threaded from the inner surface through the through hole and emerges out on the outer surface to be connected to the transmitting module.

### BRIEF DESCRIPTION OF THE DRAWINGS

Other features and advantages of the disclosure will become apparent in the following detailed description of the embodiments with reference to the accompanying drawings, of which:

FIG. 1 is a schematic perspective view illustrating a wetness notifying device mounted to a conventional diaper;

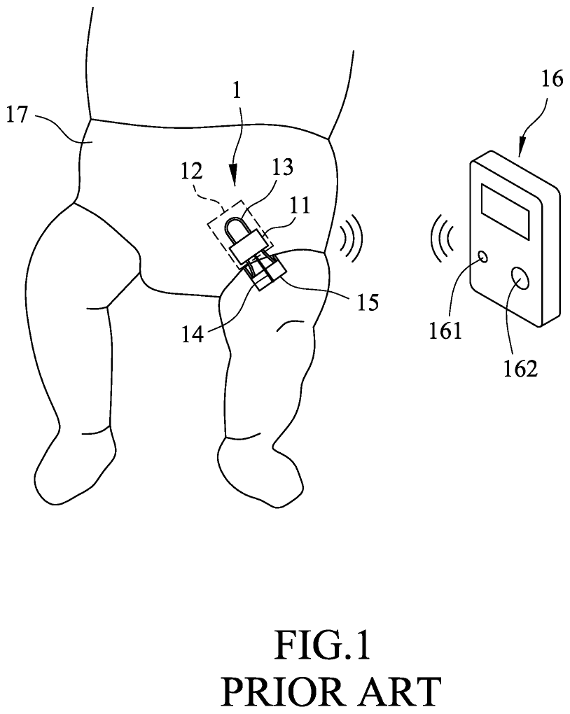

FIG. 2 is a schematic view of a diaper according to an embodiment of the present disclosure;

FIG. 3 is a schematic view of a diaper assembly including a wetness notifying device mounted to the diaper of FIG. 2 according to an embodiment of the present disclosure;

FIG. 4 is a sectional view illustrating the wetness notifying device clipping onto the diaper according to an embodiment of the present disclosure;

FIG. 5 is a schematic view of a diaper according to an embodiment of the present disclosure;

FIG. 6 is a schematic view of a diaper according to an embodiment of the present disclosure;

FIG. 7 is a schematic view of a diaper according to an embodiment of the present disclosure;

FIG. 8 is a schematic view of a diaper assembly including a wetness notifying device mounted to the diaper of FIG. 7 according to an embodiment of the present disclosure;

FIG. 9 is a schematic view of a diaper according to an embodiment of the present disclosure;

FIG. 10 is a schematic view of a diaper according to an embodiment of the present disclosure;

FIG. 11 is a schematic view of a diaper assembly according to an embodiment of the present disclosure;

FIG. 12 is a schematic view of a diaper assembly according to an embodiment of the present disclosure;

FIG. 13 is a sectional view illustrating a wetness notifying device clipping to a diaper according to an embodiment of the present disclosure; and

FIG. 14 is a sectional view illustrating the diaper assembly further including a camera device according to an embodiment of the present disclosure.

### DETAILED DESCRIPTION OF THE INVENTION

Before the disclosure is described in greater detail, it should be noted that where considered appropriate, reference numerals or terminal portions of reference numerals have been repeated among the figures to indicate corresponding or analogous elements, which may optionally have similar characteristics.

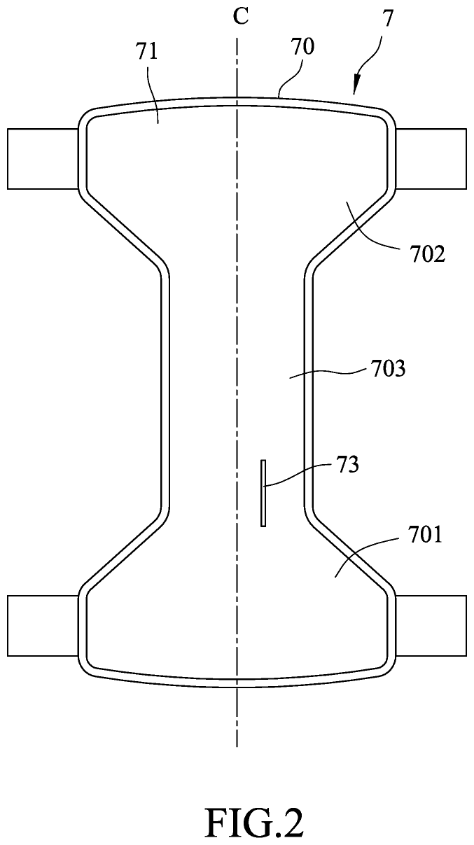

FIGS. 2 to 4 illustrate a diaper assembly according to an embodiment of this disclosure. The diaper assembly includes a wetness notifying device 10, and a diaper 7 adapted to mount the wetness notifying device 10. The diaper 7 can be detachably worn as underpants by a user 8.

The diaper 7 of the diaper assembly includes a main body 70 that is adapted to be worn as underpants by the user 8. The main body 70 includes an inner surface 71 and an outer surface 72, and is formed with a through hole 73 extending from the inner surface 71 to the outer surface 72. In some embodiments, the through hole 73 on the main body 70 is shaped in a substantially straight line that is substantially parallel to a front-rear direction (C) of the main body 70.

The wetness notifying device 10 includes a mounting module 20, a sampling unit 5, a moisture detector 22, a transmitting module 23, a transmission cable 28, and a receiving unit 4 (e.g., a smart phone). The mounting module 20 includes a sleeve body 21, a mounting seat 29 combined with the sleeve body 21, a clip 3, and a positioning member 6. The mounting seat 29 has a base plate 291 connected to the sleeve body 21.

The clip 3 is used for clipping to the main body 70, and includes a first portion 31 that is removably and partially inserted into the sleeve body 21, a second portion 32 that curvedly extends from the first portion 31, and a third portion 33 that extends from the second portion 32. When the clip 3 clips to the main body 70, the first portion 31 is disposed on the inner surface 71 of the main body 70, the second portion 32 extends through the through hole 73, and the third portion 33 is disposed on the outer surface 72 of the main body 70. The positioning member 6 engages the clip 3, and is used to secure and position the transmission cable 28. The transmission cable 28 has one end fixedly connected to the moisture detector 22, and the other end removably connected to the transmitting module 23.

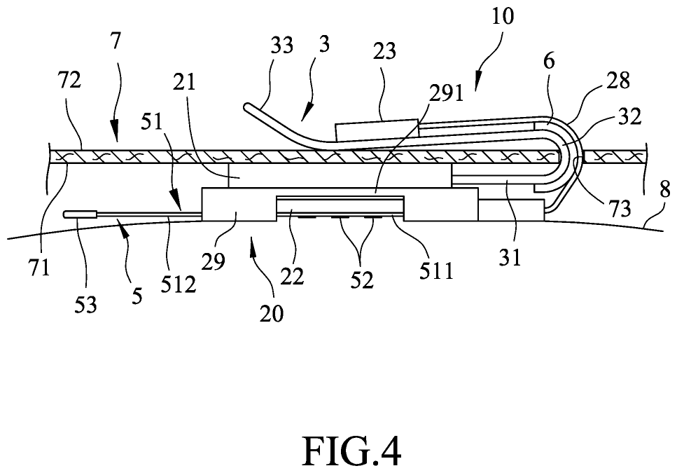

The sampling unit 5 is imbedded in the mounting seat 29, and includes a substrate 51, a plurality of sampling elements 52 (for instance, three sampling elements 52 are illustrated in FIG. 4), and a protection sleeve 53 made of silicone. The substrate 51 has a first segment 511 that is disposed in the mounting seat 29 and that is mounted with the sampling elements 52, and a second segment 512 extending from the first segment 511 out of the mounting seat 29. The protection sleeve 53 covers a distal end of the second segment 512. Each of the sampling elements 52 may be a test paper for collecting and testing urine in one embodiment, or may be a vessel for collecting urine which is to be used for subsequent analysis in other embodiments.

The moisture detector 22 is disposed on a side of the inner surface 71, and is configured to detect surrounding moisture (i.e., the moisture of a space surrounded by the diaper 7) and to output a wetness notification signal when the surrounding moisture detected thereby exceeds a threshold moisture level. Specifically, the moisture detector 22 is disposed in the mounting seat 29 and is sandwiched between the base plate 291 of the mounting seat 29 and the first segment 511 of the substrate 51, and the sampling elements 52 are disposed on a surface of the first segment 511 that faces away from the moisture detector 22.

The transmitting module 23 is disposed on a side of the outer surface 72, is electrically connected to the moisture detector 22 through the transmission cable 28 for receiving the wetness notification signal outputted by the moisture detector 22, and is configured to wirelessly transmit the wetness notification signal to the receiving unit 4. In this embodiment, the transmitting module 23 is removably attached to the third portion 33 of the clip 3. In some embodiments, the transmitting module 23 includes a first processor (not shown), a first wireless transmitter (not shown), and a battery (not shown) providing electricity to the first wireless transmitter and the first processor. The first processor receives and processes the wetness notification signal received from the moisture detector 22, and wirelessly transmits the processed wetness notification signal via the first wireless transmitter to the receiving unit 4. Upon receiving the wetness notification signal, the receiving unit 4 may output wetness notification to notify a caregiver who possesses the receiving unit 4 that the diaper 7 is wet.

In practice, the wetness notifying device 10 is mounted to the diaper 7 by making the third portion 33 of the clip 3 and the other end of the transmission cable 28 to thread, from the inner surface 71 of the main body 70, through the through hole 73 and emerging out on the outer surface 72, allowing the second portion 32 of the clip 3, the positioning member 6 and the transmission cable 28 to thread through the through hole 73. Accordingly, the third portion 33 of the clip 3 and the sleeve body 21 cooperatively grasp onto the main body 70. Subsequently, the other end of the transmission cable 28 is connected to the transmitting module 23 so that the transmission cable 28 connects the transmitting module 23 and the moisture detector 22 as illustrated in FIG. 4.

Details regarding the wetness notifying device 10 can be obtained by referring to Indian Patent Application No. 201614010143, entire content of which is incorporated herein by reference.

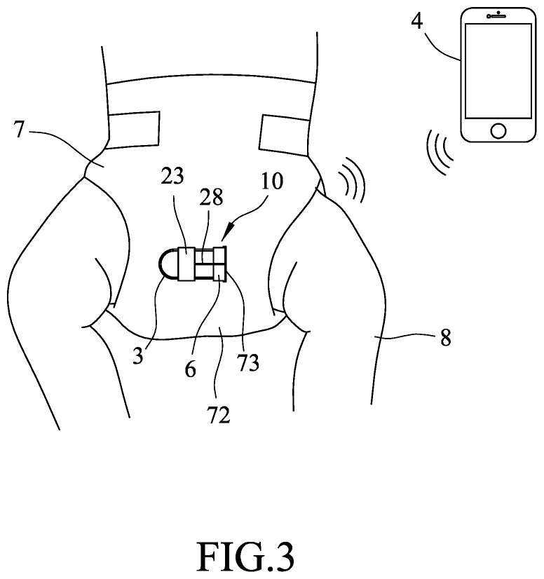

As shown in FIGS. 3 and 4, the through hole 73 on the main body 70 is configured to allow the wetness notifying device 10 to be mounted to the diaper 7 in a manner that the sampling unit 5 and the moisture detector 22 are at a position near an external urethral orifice of the user 8 when the user 8 wears the diaper 7. Accordingly, when the user 8 wearing the diaper 7 urinates and the diaper 7 becomes wet, the moisture detector 22 will be able to quickly and accurately detect wetness and send the wetness notification signal to the transmitting module 23, while simultaneously, the sampling unit 5 will test the urine. The wetness notification signal is then wirelessly transmitted to the receiving unit 4 by the transmitting module 23. Additionally, while the caregiver changes the diaper 7 for the user 8 after having received the wetness notification through the receiving unit 4, the caregiver may observe colour reaction on the sampling elements 52 and then compare the colour reaction thereon to a colour chart so as to monitor physical condition of the user 8 of the diaper 7 in real time.

Referring to FIG. 2, according to some embodiments, the main body 70 has a front part 701 for covering a lower abdomen of a wearer (such as the user 8 shown in FIG. 4) of the diaper 7, a rear part 702 for covering buttocks of the wearer, and an intermediate part 703 connected between the front part 701 and the rear part 702. The through hole 73 is formed on the intermediate part 703 at a position that is relatively closer to the front part 701 than the rear part 702, so that the sampling unit 5 and the moisture detector 22 are positioned near the external urethral orifice of the wearer when the wearer wears the diaper 7.

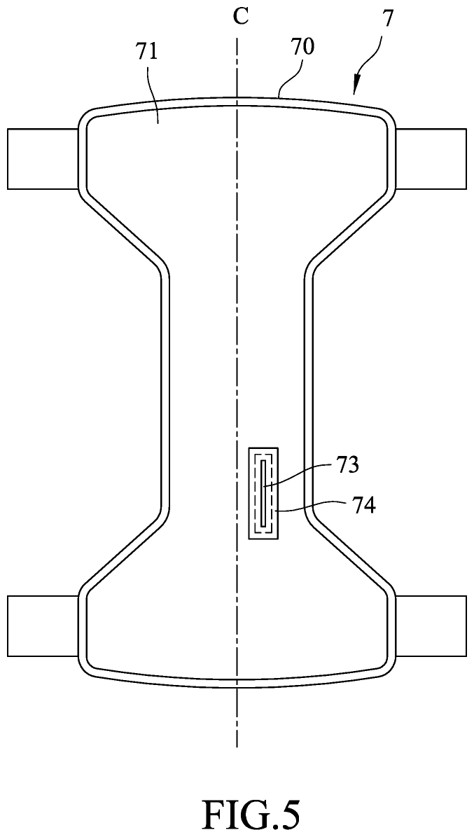

FIG. 5 shows another embodiment of the diaper 7. In this embodiment, the diaper 7 further includes a reinforcing component 74 that is attached to the main body 70 and that surrounds the through hole 73. The reinforcing component 74 enhances structural strength of the main body 70 around the through hole 73, so that the wetness notifying device 10 (see FIG. 4) is relatively stable when mounted onto the diaper 7.

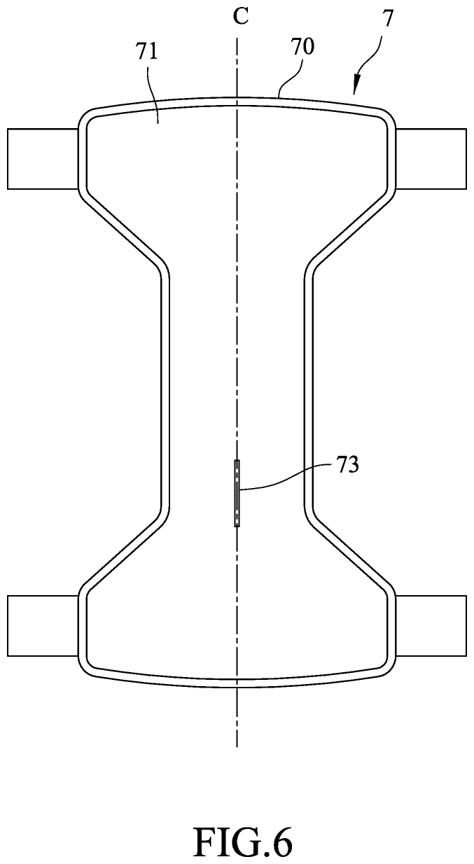

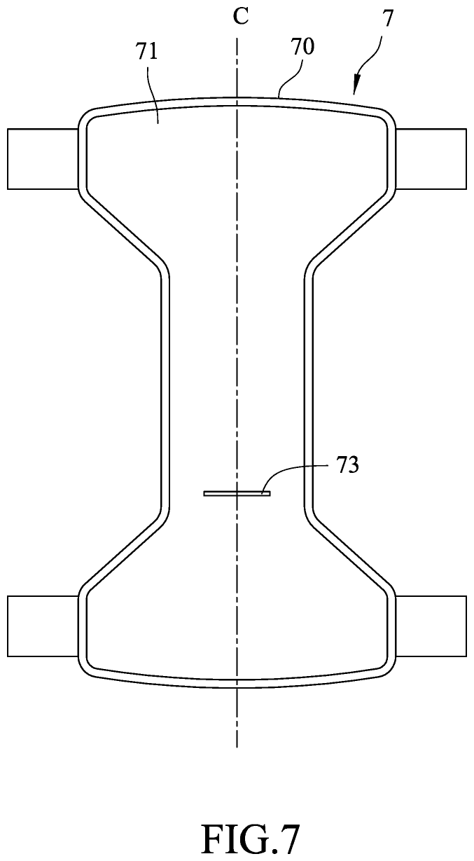

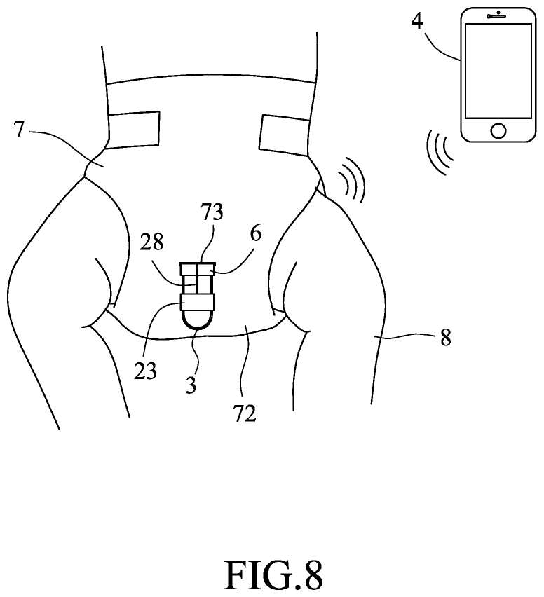

In other embodiments, the through hole 73 can take other forms. As shown in FIG. 6, the through hole 73 is shaped in a substantially straight line that is substantially parallel to the front-rear direction (C) of the main body 70, and is disposed centrally with respect to a left-right direction of the main body 70 which is perpendicular to the front-rear direction (C). In some embodiments, the through hole 73 formed on the main body 70 is substantially shaped in a straight line that is substantially perpendicular to the front-rear direction (C) of the main body 70 as illustrated in FIGS. 7 and 8.

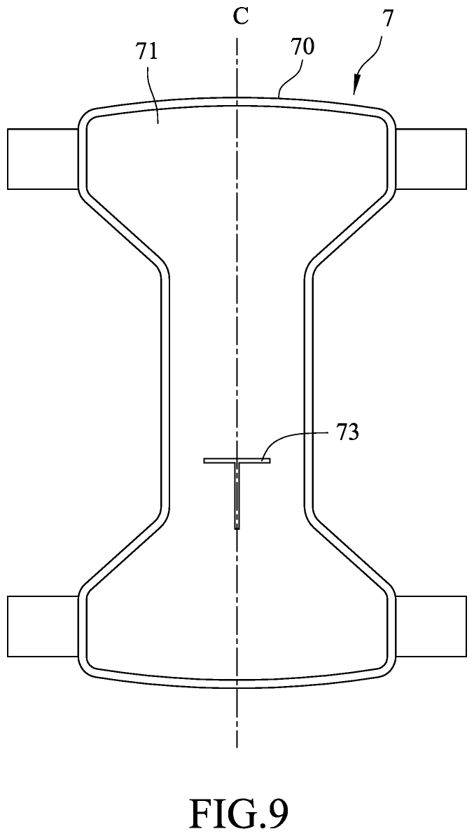

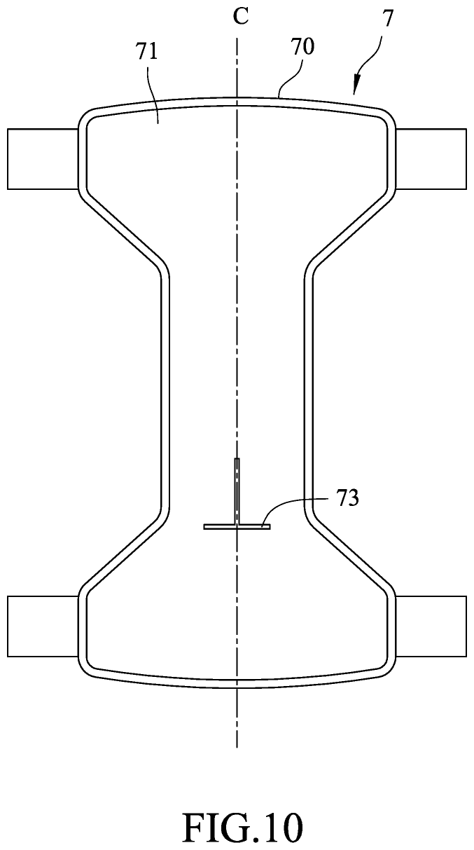

Other variations of the through hole 73 are possible. FIGS. 9 and 10 illustrate alternative embodiments of the diaper 7, in which the through hole 73 of the main body 70 substantially has a T-shape.

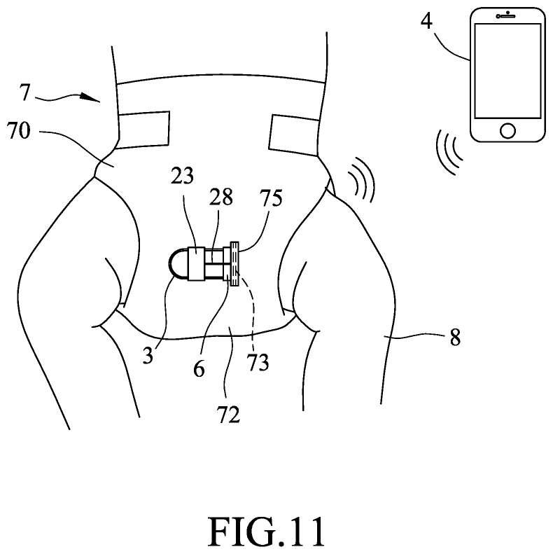

A further embodiment of the diaper 7 is illustrated in FIG. 11. In this embodiment, the diaper 7 further includes an adhesive tape 75. The through hole 73 on the main body 70 is substantially shaped in a straight line that is substantially parallel to the front-rear direction (C) of the main body 70. The adhesive tape 75 tapes the transmission cable 28 that is threaded through the through hole 73 on the main body 70 and covers the through hole 73, preventing urine from leaking out through the through hole 73.

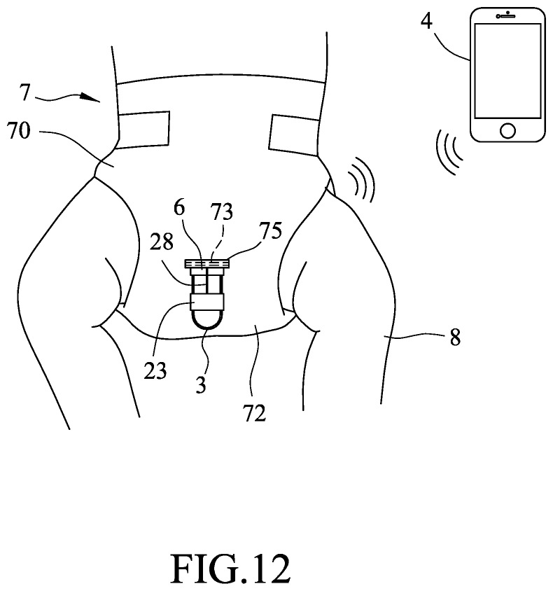

Referring to FIG. 12, another embodiment of the diaper 7 also includes the adhesive tape 75. In this embodiment, the through hole 73 is substantially shaped in a straight line that is substantially perpendicular to the front-rear direction (C) of the main body 70. The adhesive tape 75 tapes the transmission cable 28 that is threaded through the through hole 73 on the main body 70 and covers the through hole 73, preventing urine from leaking out through the through hole 73.

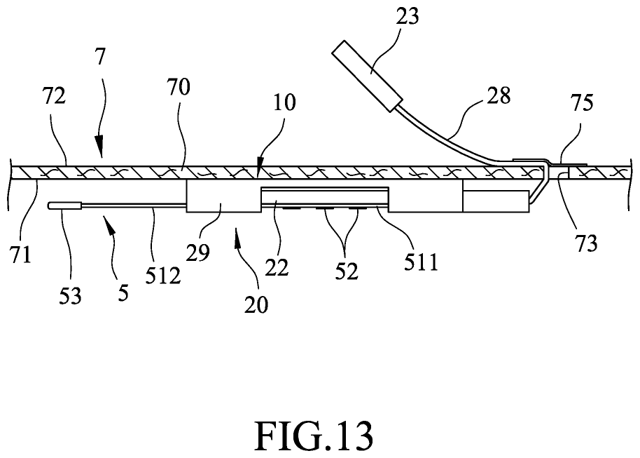

FIG. 13 illustrates a further embodiment of the diaper assembly. In this embodiment, the clip 3, the sleeve body 21 and the positioning member 6 are omitted from the wetness notifying device 10. The adhesive tape 75 is configured to tape the transmission cable 28 that is threaded through the through hole 73 on the main body 70, to cover the through hole 73, and to thereby secure the wetness notifying device 10 to the main body 70 while preventing urine from leaking out through the through hole 73.

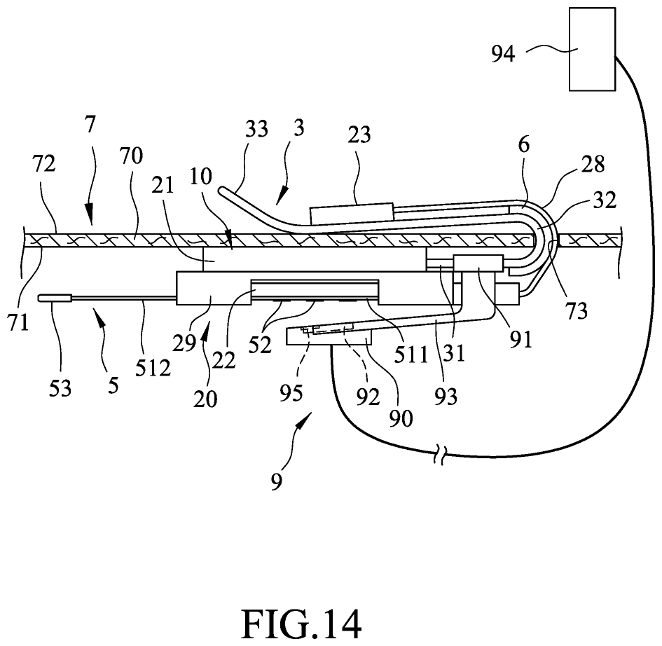

In some embodiments, the wetness notifying device 10 of the diaper assembly may further include a camera module 9. FIG. 14 illustrates an embodiment of the diaper assembly including the camera module 9. The camera module 9 includes a camera 90, a securing component 91, a connecting component 93 and a processing unit 94. The securing component 91 is secured to the first portion 31 of the clip 3, and the connecting component 93 interconnects the securing component 91 and the camera 90. The camera 90 faces the sampling elements 52 for capturing an image of the sampling elements 52, and includes a lens 92 and a light-emitting diode (LED) light 95. The LED light 95 faces the sampling elements 52, and is configured to emit light as the camera 90 captures an image of the sampling elements 52.

The processing unit 94 is electrically connected to the camera 90, and includes a second processor (not shown), a second wireless transmitter (not shown) connected to the second processor, and a battery (not shown) providing electricity to the second processor and the second wireless transmitter. The second processor is configured to process the image captured by and received from the camera 90, and to wirelessly transmit the image through the second wireless transmitter to a receiver device carried by the caregiver (e.g., a smart phone or the receiving unit 4).

With the camera module 9 included in the diaper assembly, in addition to providing wetness notification and urine test function, the wetness notifying device 10 can also provide the caregiver with images of the sampling elements 52 which are captured by the camera module 9 and transmitted wirelessly to the receiver device held by the caregiver. Accordingly, the receiver device can display the images of the sampling elements 52, and thus the caregiver can observe the colour reaction of the sampling elements 52 from the receiver device. In this way, health status of a wearer (not shown) of the diaper 7 may be remotely monitored using the receiver device (i.e., the smartphone).

Additionally, the smartphone may be installed with an application program that is configured to provide the colour chart to the caregiver. The application program may be also configured to compare the colours of the sampling elements 52 to the colour chart, and to analyse and determine the health status of the wearer based on the comparison result. The application program may store information related to comparison and analysis results. Further, the application program may present the information to the caregiver via a display of the smartphone, so that the caregiver may monitor the health status of the wearer through the application program.

In addition to the camera module 9, other devices to assist the wearer can be used in conjunction with the diaper assembly. It should be especially noted that in various embodiments, the through hole 73 of the main body 70, in addition to allowing the transmission cable 28 to be threaded therethrough, may also allow the threading through of a urinary catheter or the insertion of a urine collection bag. That is to say, the through hole 73 may provide an outlet for the urinary catheter to drain the wearer's urine. Although the wetness notifying device 10 of the diaper assembly is designed to detect wetness from urination by the wearer, it is not limited to detecting wetness from urine sources. The wetness notifying device 10 may also detect wetness from faeces defecated by the wearer, and the caregiver will be given notification about the defecation.

Additionally, the through hole 73 on the main body 70 may allow the insertion of a cleaning tube, which allows the caregiver to use water, disinfectant or other cleaning solutions to carry out preliminary cleaning of the wearer's urine and/or faeces.

In summary, the through hole 73 on the main body 70 of the diaper 7 allows the moisture detector 22 and the sampling unit 5 to be positioned near the external urethral orifice of the wearer. Accordingly, when the wearer urinates and wets the diaper 7, the wetness notifying device 10 will quickly detect the wetness and send a wetness notification signal to the receiving unit 4 (e.g., a smartphone), thus allowing the caregiver to receive the wetness notification and to monitor the health status of the wearer using the smartphone.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

I claim:

1. A diaper (7) adapted to mount a wetness notifying device (10) that includes a moisture detector (22) configured to detect surrounding moisture and to output a notification signal when the surrounding moisture detected thereby exceeds a threshold moisture level, a transmission cable (28), and a transmitting module (23) that is electrically connected to the moisture detector (22) through the transmission cable (28) for receiving the notification signal outputted by the moisture detector (22) and that is configured to wirelessly transmit the notification signal, the transmission cable (28) having one end fixedly connected to the moisture detector (22) and the other end removably connected to the transmitting module (23), said diaper (7) being characterized by:
a main body (70) that is adapted to be worn as underpants by a user (8), that includes an inner surface (71) and an outer surface (72), and that is formed with a through hole (73) extending from said inner surface (71) to said outer surface (72) and being configured to allow the wetness notifying device (10) to be mounted to said diaper (7) in a manner that the moisture detector (22) is disposed on a side of said inner surface (71) and the transmitting module (23) is disposed on a side of said outer surface (72), that the other end of the transmission cable (28) is threaded from said inner surface (71) through said through hole (73) and emerges out on said outer surface (72) and is connected to the transmitting module (23), and that the moisture detector (22) is at a position near an external urethral orifice of the user (8) wearing said diaper (7).

2. The diaper (7) as claimed in claim 1, wherein said through hole (73) is substantially shaped in a straight line.

3. The diaper (7) as claimed in claim 1, wherein said through hole (73) is substantially shaped in a straight line that is substantially parallel to a front-rear direction (C) of said main body (70).

4. The diaper (7) as claimed in claim 1, wherein said through hole (73) is substantially shaped in a straight line that is substantially perpendicular to a front-rear direction (C) of said main body (70).

5. The diaper (7) as claimed in claim 1, wherein said through hole (73) substantially has a T-shape.

6. The diaper (7) as claimed in claim 1, characterized by a reinforcing component (74) that is attached to said main body (70) and that surrounds said through hole (73).

7. The diaper (7) as claimed in claim 1, characterized by an adhesive tape (75) configured to tape the transmission cable (28) that is threaded through said through hole (73) on said main body (70) and to cover said through hole (2).

8. A diaper assembly being characterized by:
a diaper (7) including a main body (70) that is adapted to be worn as underpants by a user (8), that includes an inner surface (71) and an outer surface (72), and that is formed with a through hole (73) extending from said inner surface (71) to said outer surface (72); and
a wetness notifying device (10) including
a moisture detector (22) disposed on a side of said inner surface (71) at a position near an external urethral orifice of the user (8) wearing said diaper (7), and configured to detect surrounding moisture and to output a notification signal when the surrounding moisture detected thereby exceeds a threshold moisture level,
a transmitting module (23) disposed on a side of said outer surface (72), electrically connected to said moisture detector (22) for receiving the notification signal outputted by said moisture detector (22), and configured to wirelessly transmit the notification signal, and
a transmission cable (28) having one end fixedly connected to said moisture detector (22) and the other end removably connected to said transmitting module (23), said the other end of the transmission cable (28) being threaded from said inner surface (71) through said through hole (73) and emerging out on said outer surface (72) to be connected to said transmitting module (23).

9. The diaper assembly as claimed in claim 8, wherein said through hole (73) is substantially shaped in a straight line.

10. The diaper assembly as claimed in claim 8, wherein said through hole (73) is substantially shaped in a straight line that is substantially parallel to a front-rear direction (C) of said main body (70).

11. The diaper assembly as claimed in claim 8, wherein said through hole (73) is substantially shaped in a straight line that is substantially perpendicular to a front-rear direction (C) of said main body (70).

12. The diaper assembly as claimed in claim 8, wherein said through hole (73) substantially has a T-shape.

13. The diaper assembly as claimed in claim 8, characterized by a reinforcing component (74) that is attached to said main body (70) and that surrounds said through hole (73).

14. The diaper assembly as claimed in claim 8, characterized by an adhesive tape (75) taping said transmission cable (28) that is threaded through said through hole (73) on said main body (70) and covering said through hole (73).
